(19) United States
(12) Patent Application Publication
(10) Pub. No.: US 2024/0379220 A1
(43) Pub. Date: Nov. 14, 2024

(54) SYSTEMS AND METHODS FOR PERFORMING STRUCTURAL EQUATION MODELING (SEM) USING HEALTHCARE PRODUCT DATA

(71) Applicant: Optum, Inc., Minnetonka, MN (US)

(72) Inventors: Neha CHADDHA, Dwarka (IN); Biswajit BHATTACHARJEE, Noida (IN); Tarun MINOCHA, Gurgaon (IN); Kamlesh KUMAR, Bangalore (IN); Krishan KUMAR, Aligarh (IN)

(21) Appl. No.: 18/315,550

(22) Filed: May 11, 2023

(51) Int. Cl. G16H 40/63

(52) U.S. Cl. CPC G16H 40/63

(57) ABSTRACT

Systems and methods for determining relationships between key performance indicators (KPIs) associated with a healthcare product are disclosed. A method includes receiving information identifying a set of KPIs associated with a healthcare product, determining a hierarchy of the set of KPIs, determining directional effects between the set of KPIs, based on the hierarchy, determining, using structural equation modeling, values of the directional effects, providing the values of the directional effects, and performing a resource allocation action based on the values of the directional effects.

### TECHNICAL FIELD

The present disclosure relates to systems and methods for determining relationships between key performance indicators (KPIs) associated with a healthcare product, utilizing a structural equation modeling (SEM) technique.

### BACKGROUND

Healthcare product data can be modeled using various types of modeling approaches. For instance, healthcare product data can be modeled using a regression technique. Regression techniques generally examine relations and estimate the direct impact of multiple predictors on a single dependent variable. Moreover, regression techniques generally assume that the collected data is error free and of perfect measurement. Further still, regression techniques generally only estimate the direct impact of a single variable on another variable.

As another example, graph modeling permits a model to be generated for a complete system by fitting a sequence of sub-models. However, when using graph modeling techniques, it is difficult to explicitly adjust for measurement error without introducing a separate stage of latent variable modeling. Further, temporal ordering of the variables is generally required using graph modeling techniques.

Accordingly, a need exists for a more accurate modeling technique for healthcare product data.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section

### SUMMARY

According to some embodiments, a computer-implemented method includes receiving, by one or more processors, information identifying a set of key performance indicators (KPIs) associated with a healthcare product; determining, by the one or more processors, a hierarchy of the set of KPIs; determining, by the one or more processors, directional effects between the set of KPIs, based on the hierarchy; determining, by the one or more processors and using structural equation modeling, values of the directional effects; providing, by the one or more processors, the values of the directional effects; and performing, by the one or more processors, a resource allocation action based on the values of the directional effects.

According to some embodiments, a device includes a memory configured to store instructions; and one or more processors configured to execute the instructions to perform operations comprising: receiving information identifying a set of key performance indicators (KPIs) associated with a healthcare product; determining a hierarchy of the set of KPIs; determining directional effects between the set of KPIs, based on the hierarchy; determining, using structural equation modeling, values of the directional effects; providing the values of the directional effects; and performing a resource allocation action based on the values of the directional effects.

According to some embodiments, a non-transitory computer-readable medium stores instructions that, when executed by one or more processors, cause the one or more processors to perform operations comprising: receiving information identifying a set of key performance indicators (KPIs) associated with a healthcare product; determining a hierarchy of the set of KPIs; determining directional effects between the set of KPIs, based on the hierarchy; determining, using structural equation modeling, values of the directional effects; providing the values of the directional effects; and performing a resource allocation action based on the values of the directional effects

SEM refers to a statistical technique based on a linear equation system used to examine causal relationships between two or more variables. The embodiments herein can utilize SEM and healthcare product data to generate more accurate models, and to more accurately identify interrelationships between KPIs associated with the healthcare product data.

It can be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 is a diagram of an example system for performing SEM using healthcare product data.

FIG. 2 is a diagram of example components of one or more devices of FIG. 1.

FIG. 3 is a flowchart of an example process for performing SEM using healthcare product data.

FIG. 4 is a diagram of an example hierarchy of a set of KPIs.

FIG. 5 is a diagram of example directional effects between the set of KPIs.

FIGS. 6A and 6B are diagrams of example values of the directional effects between the set of KPIs.

FIG. 7 is a diagram of example values of directional effects between the set of KPIs.

FIG. 8 is a diagram of example values of directional effects between the set of KPIs.

### DETAILED DESCRIPTION

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

Various non-limiting embodiments of the present disclosure will now be described to provide an overall understanding of the principles of the structure, function, and use of systems and methods disclosed herein for determining relationships between KPIs associated with a healthcare product, utilizing an SEM technique.

As addressed above, a need exists for a more accurate modeling technique for healthcare product data. Some embodiments solve this technical problem by applying SEM techniques to healthcare data to determine interrelationships between KPIs and values of directional effects of such interrelationships between the KPIs. For instance, some embodiments receive KPIs and determine a hierarchy between the KPIs. Further, some embodiments determine directional effects between the KPIs and determine, using SEM, values of the directional effects. Some embodiments provide the values of the directional effects and perform a resource allocation action based on the values

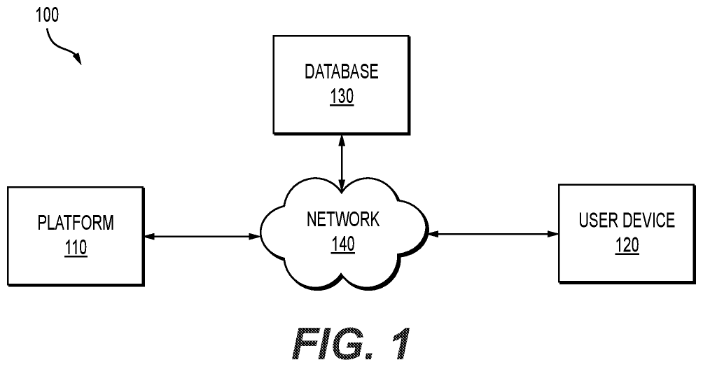

FIG. 1 is a diagram of an example system 100 for performing SEM using healthcare product data. As shown in FIG. 1, the system 100 can include a platform 110, a user device 120, a database 130, and a network 140.

The platform 110 can include a device configured to receive information identifying a set of KPIs associated with a healthcare product from the user device 120, based on an input received via a graphical user interface (GUI) of the user device 120; determine a hierarchy of the set of KPIs; determine directional effects between the set of KPIs, based on the hierarchy; determine, using structural equation modeling, values of the directional effects; and provide the values of the directional effects to the user device 120 to cause the user device 120 to display the values of the directional effects via the GUI of the user device. For example, the platform 110 can be a cloud server, a server, a computer, or the like.

The user device 120 can include a device configured to receive information identifying a set of KPIs associated with a healthcare product via a GUI of the user device 120; provide the information identifying the set of KPIs associated with the healthcare product to the platform 110; receive values of directional effects between the set of KPIs from the platform 110; and display the values of the directional effects via the GUI. For example, the user device 120 can be a smartphone, a desktop computer, a laptop computer, a wearable device, or the like.

The database 130 can include a device configured to store healthcare product data, information identifying a set of KPIs, values of directional effects between the set of KPIs, or the like. For example, the database 130 can be a cloud database, a centralized database, a commercial database, a distributed database, or the like.

The network 140 can include a network that permits communication between the platform 110, the user device 120, and/or the database 130. For example, the network 140 can be a cellular network (e.g., a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, or the like, and/or a combination of these or other types of networks.

The number and arrangement of the devices of the system 100 shown in FIG. 1 are provided as an example. In practice, the system 100 can include additional devices, fewer devices, different devices, or differently arranged devices than those shown in FIG. 1. Additionally, or alternatively, a set of devices (e.g., one or more devices) of the system 100 can perform one or more functions described as being performed by another set of devices of the system 100.

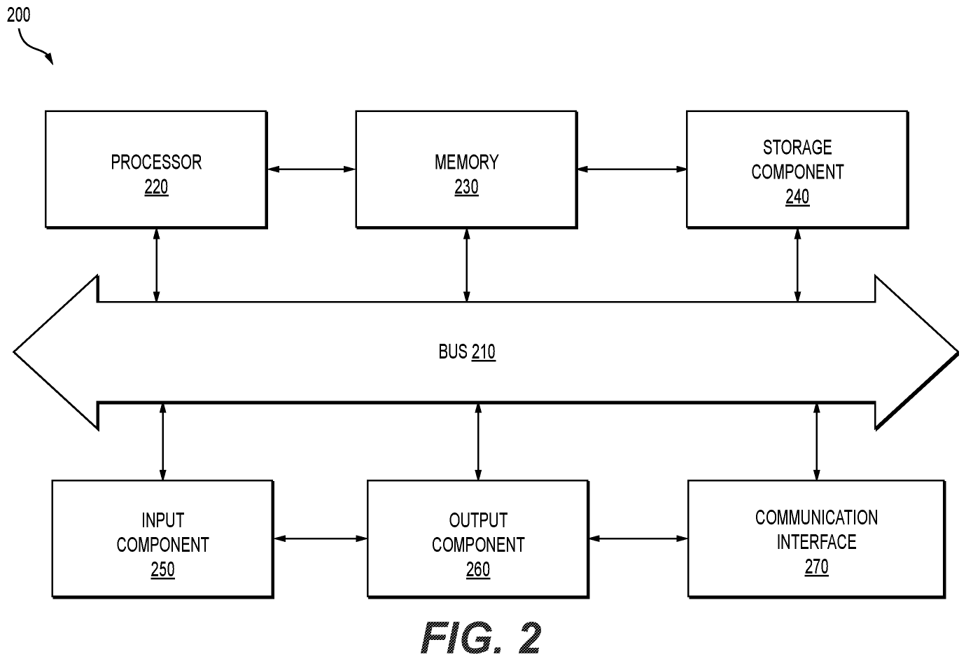

FIG. 2 is a diagram of example components of one or more devices of FIG. 1. The device 200 can correspond to the platform 110, the user device 120, and/or database 130. As shown in FIG. 2, the device 200 can include a bus 210, a processor 220, a memory 230, a storage component 240, an input component 250, an output component 260, and a communication interface 270.

The bus 210 includes a component that permits communication among the components of the device 200. The processor 220 can be implemented in hardware, firmware, or a combination of hardware and software. The processor 220 can be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 220 can include one or more processors capable of being programmed to perform a function. The memory 230 can include a random access memory (RAM), a read only memory (ROM), and/or another type of dynamic or static storage device (e.g., a flash memory, a magnetic memory, and/or an optical memory) that stores information and/or instructions for use by the processor 220.

The storage component 240 can store information and/or software related to the operation and use of the device 200. For example, the storage component 240 can include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 250 can include a component that permits the device 200 to receive information, such as via user input (e.g., a touch screen display, a keyboard, a keypad, a mouse, a button, a switch, and/or a microphone for receiving the reference sound input). Additionally, or alternatively, the input component 250 can include a sensor for sensing information (e.g., a global positioning system (GPS) component, an accelerometer, a gyroscope, and/or an actuator). The output component 260 can include a component that provides output information from the device 200 (e.g., a display, a speaker for outputting sound at the output sound level, and/or one or more light-emitting diodes (LEDs)).

The communication interface 270 can include a transceiver-like component (e.g., a transceiver and/or a separate receiver and transmitter) that enables the device 200 to communicate with other devices, such as via a wired connection, a wireless connection, or a combination of wired and wireless connections. The communication interface 270 can permit the device 200 to receive information from another device and/or provide information to another device. For example, the communication interface 270 can include an Ethernet interface, an optical interface, a coaxial interface, an infrared interface, a radio frequency (RF) interface, a universal serial bus (USB) interface, a Wi-Fi interface, a cellular network interface, or the like.

The device 200 can perform one or more processes described herein. The device 200 can perform these processes based on the processor 220 executing software instructions stored by a non-transitory computer-readable medium, such as the memory 230 and/or the storage component 240. A computer-readable medium can be defined herein as a non-transitory memory device. A memory device can include memory space within a single physical storage device or memory space spread across multiple physical storage devices.

The software instructions can be read into the memory 230 and/or the storage component 240 from another computer-readable medium or from another device via the communication interface 270. When executed, the software instructions stored in the memory 230 and/or the storage component 240 can cause the processor 220 to perform one or more processes described herein. Additionally, or alternatively, hardwired circuitry can be used in place of or in combination with software instructions to perform one or more processes described herein. Thus, implementations described herein are not limited to any specific combination of hardware circuitry and software.

The number and arrangement of the components shown in FIG. 2 are provided as an example. In practice, the device 200 can include additional components, fewer components, different components, or differently arranged components than those shown in FIG. 2. Additionally, or alternatively, a set of components (e.g., one or more components) of the device 200 can perform one or more functions described as being performed by another set of components of the device 200.

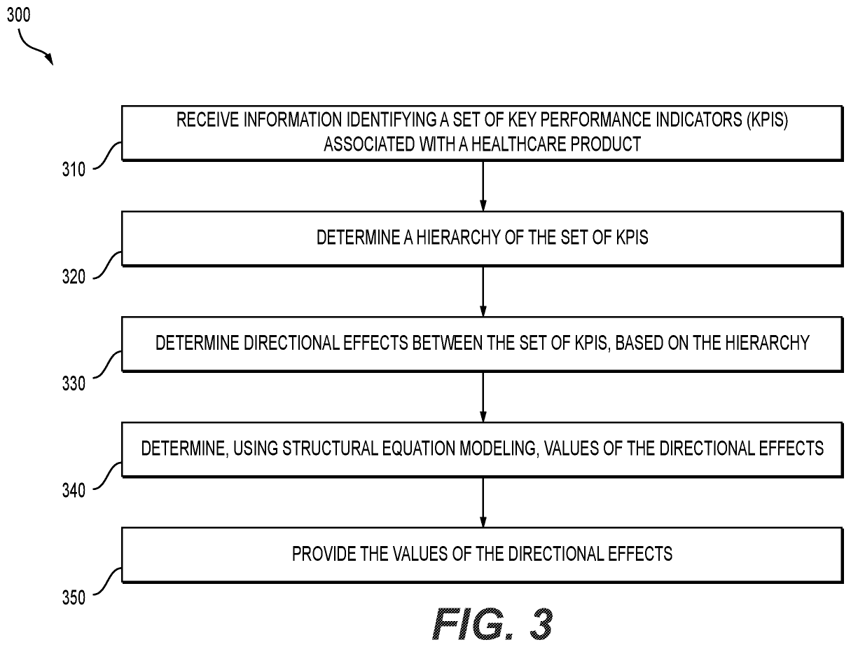

FIG. 3 is a flowchart of an example process for performing SEM using healthcare product data.

As shown in FIG. 3 at block 310, the process 300 can include receiving information identifying a set of key performance indicators (KPIs) associated with a healthcare product. For example, the platform 110 can receive information identifying a set of KPIs associated with a healthcare product from the user device 120, based on an input received via a GUI of the user device 120.

In some implementations, a user of the user device 120 can input the information identifying the set of KPIs via the GUI of the user device 120, and cause the user device 120 to provide the information identifying the set of KPIs to the platform 110. In this case, the platform 110 can receive the information identifying the set of KPIs from the user device 120, based on the input received via the GUI of the user device 120.

The healthcare product can be a product offered by an entity (e.g., a healthcare company, an insurance company, or the like). For example, the healthcare product can be a Medicare Advantage plan, a Medicare supplemental insurance plan, a dual eligible special need plan, an individual and family plan, or the like. The entity can market the healthcare product via different channels. For example, a channel can be a paid search, an affiliate, a display, social media, email, website, a referral via another entity, or the like. The entity can receive applications for the healthcare product that originate from various channels. In this way, an application for the healthcare product can be attributed to a particular channel.

In some implementations, the KPIs can include online spend, offline spend, visits, tool visits, started applications, completed applications, leads, switch calls, approved applications, and attributed applications. However, it should be understood that the embodiments herein are applicable to other types of KPIs.

“Online spend” can refer to spend associated with online marketing for the healthcare product. “Offline spend” can refer to spend associated with offline marketing for the healthcare product. “Visits” can refer to a number visits to a website associated with the healthcare product. “Tool visits” can refer to a number of visits to a website where the visitor interacted with one or more tools provided by, or otherwise accessible via, the website. “Started applications” can refer to a number of applications for the healthcare product that were started. “Completed applications” can refer to a number of applications for the healthcare product that were completed. “Leads” can refer to a number of forms requesting additional information associated with the healthcare product that were submitted to the entity, a number of product requests, a number of agent appointments, a number of education requests, etc. “Switch calls” can refer to a number of calls provided to a call center associated with the entity. “Approved applications” can refer to a number of applications for the healthcare product that are approved by the entity. “Attributed applications” can refer to a number of applications for the healthcare product that are attributed to a particular channel.

As shown in FIG. 3 at block 320, the process 300 can include determining a hierarchy of the set of KPIs. For example, the platform 110 can determine a hierarchy of the set of KPIs.

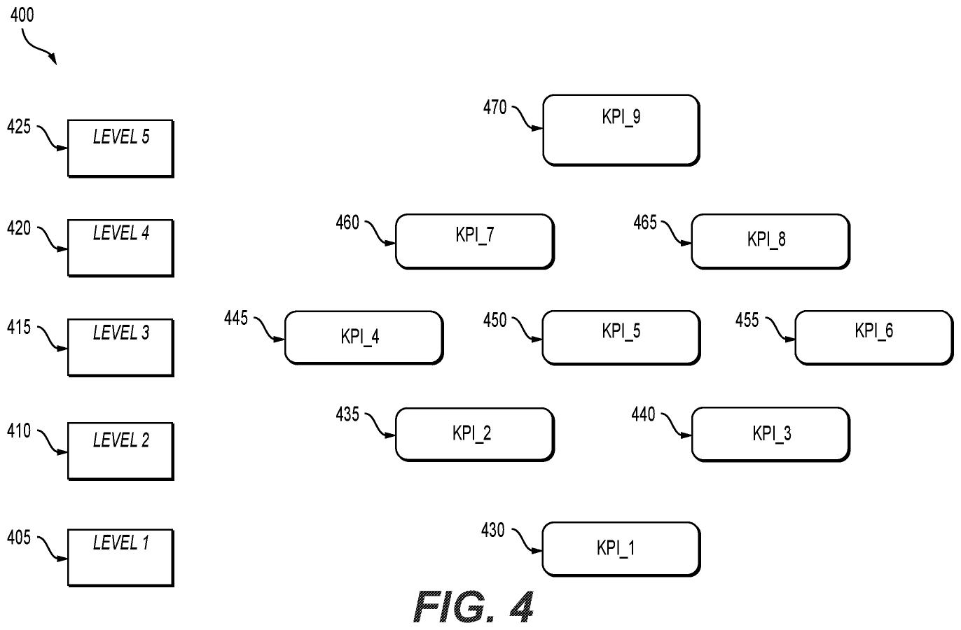

The “hierarchy” can refer to a hierarchical relationship between the KPIs. For instance, the hierarchy can include a set of levels. For instance, as shown in FIG. 4, which is a diagram 400 of an example hierarchy of a set of KPIs, the hierarchy can include a set of levels, such as a first level 405, a second level 410, a third level 415, a fourth level 420, and a fifth level 425. As shown, the first level 405 can include the KPI_1 430 (e.g., spend), the second level 410 can include the KPI_2 435 (e.g., visits) and the KPI_3 440 (e.g., tool visits), the third level 415 can include the KPI_4 445 (e.g., leads), the KPI_5 450 (e.g., completed applications), and the KPI_6 455 (e.g., started applications), the fourth level 420 can include the KPI_7 460 (e.g., approved applications) and the KPI_8 465 (e.g., switch calls), and the fifth level 425 can include the KPI_9 470 (e.g., attributed applications).

The KPIs in a same level can affect each other. For instance, and referring to FIG. 4, the KPI_2 435 in the second level 410 can affect the KPI_2 435 in the second level 410. The KPIs in a lower level can affect the KPIs in a higher level. For instance, and referring to FIG. 4, the KPI_1 430 provided in the first level 405 can affect the KPI_2 435 and the KPI_3 440 in the second level 410. The KPIs in a higher level might not affect the KPIs in a lower level. For instance, and referring to FIG. 4, the KPI_2 435 and the KPI_3 440 in the second level 410 might not affect the KPI_1 430 provided in the first level 405.

In some implementations, the platform 110 can determine the hierarchy of the set of KPIs based on an input received via the GUI of the user device 120. For example, a user of the user device 120 can input information identifying the hierarchy of the set of KPIs, and the platform 110 can determine the hierarchy based on the input. Alternatively, the platform 110 can determine the hierarchy of the set of KPIs using a model (e.g., a machine learning model). For example, the platform 110 can input information identifying the set of KPIs into the model, and determine the hierarchy based on an output of the model. As another alternative, the platform 110 can receive information identifying the hierarchy from the database 130, and determine the hierarchy based on the information received from the database 130.

As shown in FIG. 3 at block 330, the process 300 can include determining directional effects between the set of KPIs, based on the hierarchy. For example, the platform 110 can determine directional effects between the set of KPIs, based on the hierarchy.

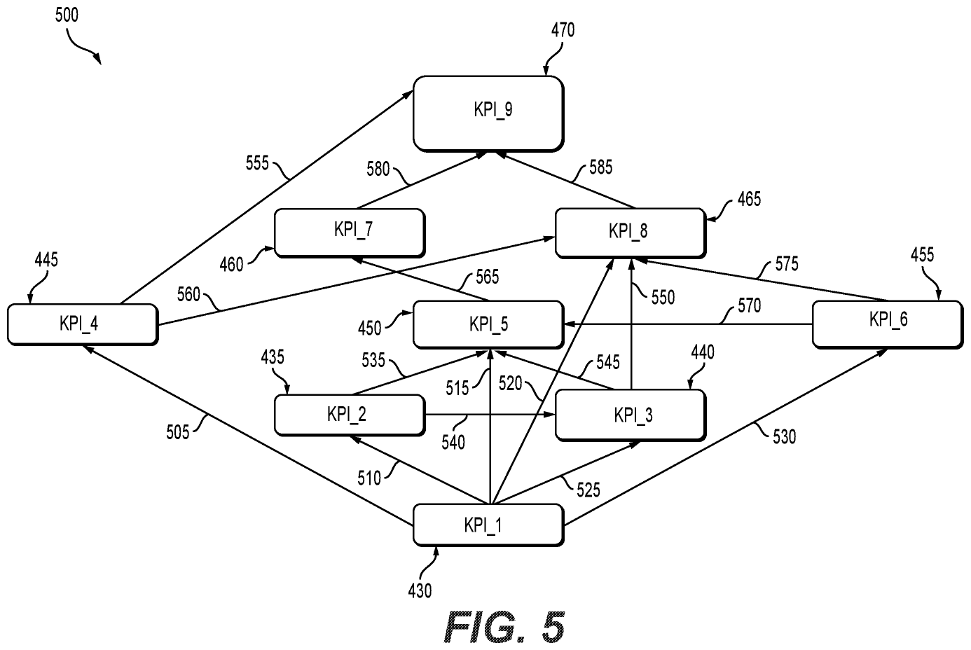

A “directional effect” can refer to an effect of a first KPI on a second KPI. In some implementations, a directional effect can be a direct effect. For instance, and referring to FIG. 5, which is a diagram 500 of example directional effects between the set of KPIs, the KPI_1 430 can include a directional effect 505 on the KPI_4 445, can include a directional effect 510 on the KPI_2 435, can include a directional effect 515 on the KPI_5 450, can include a directional effect 520 on the KPI_8 465, can include a directional effect 525 on the KPI_3 440, and can include a directional effect 530 on the KPI_6 455.

As another example, the KPI_2 435 can include the directional effect 535 on the KPI_5 450, and can include the directional effect 540 on the KPI_3 440. As another example, the KPI_3 440 can include the directional effect 545 on KPI_5 450, and can include the directional effect 550 on the KPI_8 465. As another example, the KPI_4 445 can include the directional effect 555 on the KPI_9 470, and can include the directional effect 560 on the KPI_8 465. As another example, the KPI_5 450 can include the directional effect 565 on the KPI_7 460. As another example, the KPI_6 455 can include the directional effect 570 on the KPI_5 450, and can include the directional effect 575 on the KPI_8 465. As another example, the KPI_7 460 can include the directional effect 580 on the KPI_9 470. As another example, the KPI_8 465 can include the directional effect 585 on the KPI_9 470.

Additionally, or alternatively, a directional effect can be an indirect effect. As an example, the KPI_1 430 can include the directional effect 555, which is an indirect effect, on the KPI_9 470. In other words, the KPI_1 430 includes a direct directional effect 505 on the KPI_4 445, and includes an indirect directional effect, via the directional effect 555, on the KPI_9 470.

In some implementations, the platform 110 can determine the directional effects between the set of KPIs based on an input received via the GUI of the user device 120. For example, a user of the user device 120 can input information identifying the directional effects between the set of KPIs, and the platform 110 can determine the directional effects between the set of KPIs based on the input. Alternatively, the platform 110 can determine the directional effects between the set of KPIs using a model (e.g., a machine learning model). For example, the platform 110 can input information identifying the set of KPIs and the hierarchy of the set of KPIs into the model, and determine the directional effects between the set of KPIs based on an output of the model. As another alternative, the platform 110 can receive information identifying the directional effects between the set of KPIs from the database 130, and determine the directional effects between the set of KPIs based on the information received from the database 130.

As shown in FIG. 3 at block 340, the process 300 can include determining, using structural equation modeling, values of the directional effects. For example, the platform 110 can determine, using SEM, values of the directional effects. As a particular example, using SEM, the platform 110 can set each KPI as a target variable, and simultaneously perform multiple regressions with multiple observed variables to determine values of the directional effects.

The platform 110 can receive training data associated with the KPIs, and can determine the values of the directional effects using SEM and the training data. In this case, the training data can include historical information associated with the healthcare product. For example, the training data can identify a number of attributed applications, an amount of online spend, an amount of offline spend, a number of visits, a number of tool visits, a number of started applications, a number of completed applications, a number of leads, a number of switch calls, a number of approved applications, or the like.

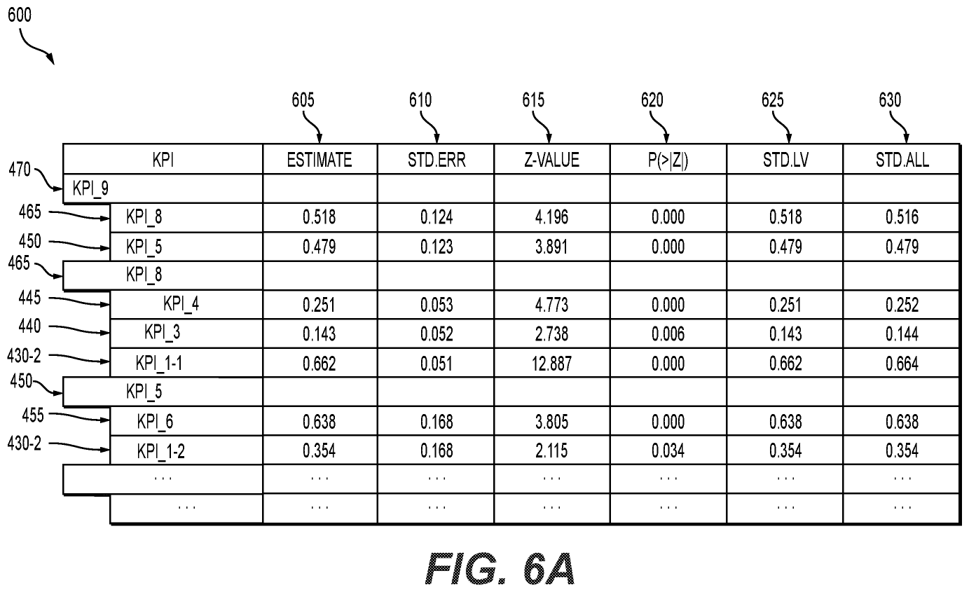

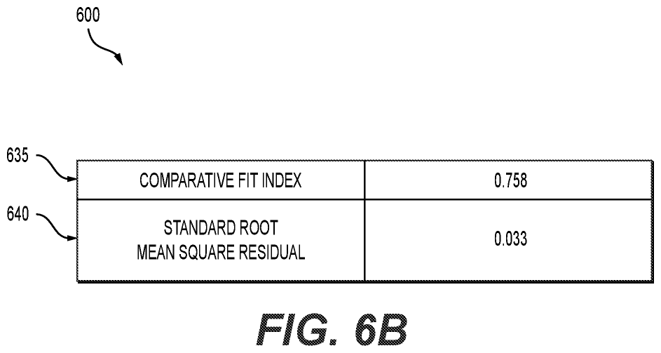

A “value” of a directional effect can identify a magnitude of the directional effect. Or, put another way, a “value” of a directional effect between two KPIs can identify the extent of impact of a first KPI on the second KPI. In some implementations, the value can be an estimate, a standard error, a z-value, a p-value, a standardized estimate of a latent variable, a standardized estimate, a comparative fit index, a standardized root mean square residual, or the like. For example, and referring to FIGS. 6A and 6B, which are diagrams 600 of example values of the directional effects between the set of KPIs, the platform 110 can determine, for each of the KPIs 430 through 470, estimates 605, standard errors 610, z-values 615, p-values 620, standardized estimates of latent variables 625, and standardize estimates 630. Moreover, as shown in FIG. 6B, the platform 110 can determine a comparative fit index 635 and a standardized root mean square residual 640 based on values of the directional effects.

An “estimate” (or a “coefficient”) can refer to a change in a dependent variable associated with a unit change or a standard deviation change in an independent variable. For example, and referring to FIG. 6A, the platform 110 can determine a value of a directional effect of the KPI_8 465 on the KPI_9 470 as being “0.518.” In this case, a unit change or a standard deviation change in the KPI_8 465 can have a change of 0.518 in the KPI_9 470. As another example, the platform 110 can determine a value of a directional effect of the KPI_5 450 on the KPI_9 470 as being “0.479.” In this case, a unit change or a standard deviation change in the KPI_5 450 can have a change of 0.479 in the KPI_9 470. A “standard error” can refer to a standard deviation of an estimate's sampling distribution. For instance, the standard error can reflect a variability around an estimated regression line and an accuracy of a regression model. A “z-value” can refer to an estimate divided by a standard error. A “p-value” can refer to a statistical measurement used to validate a hypothesis against observed data. A “standardized estimate of latent variables” can refer to standardized estimates for latent variables. A “standardized estimate” can refer to a standardized estimate. A “comparative fit index” can refer to a measure of how well a model fits to particular data. “Standardized root mean square residual” can refer to error or unexplained variance of the variables.

In some implementations, the platform 110 can determine one or more of the foregoing values, and determine whether the one or more values satisfy respective thresholds. “Satisfy a threshold” can refer to a value being greater than a threshold, less than a threshold, greater than or equal to a threshold, or less than or equal to a threshold.

In some implementation, the platform 110 can determine an updated hierarchy of the set of KPIs and/or determine updated directional effects between the set of KPIs, based on determining that one or more values satisfy a threshold. For example, the platform 110 can determine whether a p-value of a first KPI having a directional effect on a second KPI is less than a threshold of, as an example, 0.05. If the platform 110 determines that the p-value is greater than the threshold, then the platform 110 can determine an updated directional hierarchy and/or updated directional effect in which the first KPI is not shown as affecting the second KPI. As another example, the platform 110 can determine whether a comparative fit index is greater than, as an example, 0.75. If the comparative fit index is greater than 0.75, then the platform 110 can determine that the hierarchy and/or the directional effects are validated.

As shown in FIG. 3 at block 340, the process 300 can include providing the values of the directional effects. For example, the platform 110 can provide the values of the directional effects to the user device 120 to permit the user device 120 to display the values of the directional effects via a user interface.

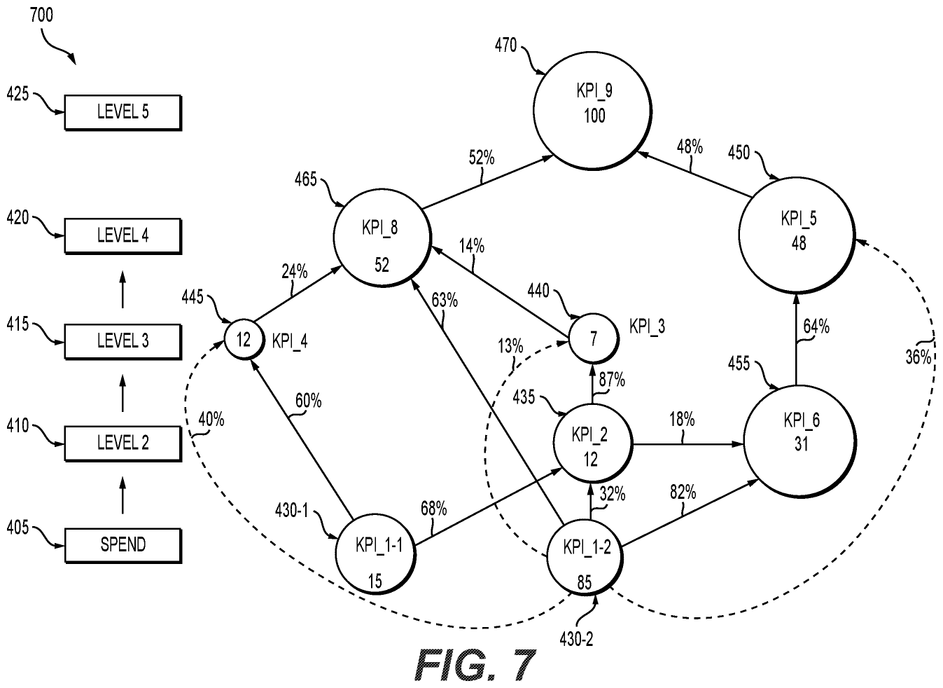

As an example, and as shown in FIG. 7, which is a diagram 700 of example values of directional effects between the set of KPIs, the user device 120 can display the diagram 700 which includes the KPIs 430 through 470, and values of the directional effects between the KPIs. As an example, and as shown, the KPI_4 445 can be directly affected by the KPI_1-1 430-1 and the KPI_1-2 430-2. In this case, the KPI_1-1 430-1 can have a value of 0.6 of a directional effect on the KPI_4 445, and the KPI_1-2 430-2 can have a value of 0.4 of a directional effect on the KPI_4 445. Moreover, as shown in FIG. 7, the diagram 700 can include indicators of how many of the KPI_9 470 were attributable to each KPI. For instance, as an example, 85 of 100 attributed applications can be attributed to the KPI_1-2 430-2, whereas 15 of 100 attributed applications can be attributable to the KPI_1-1 430-1.

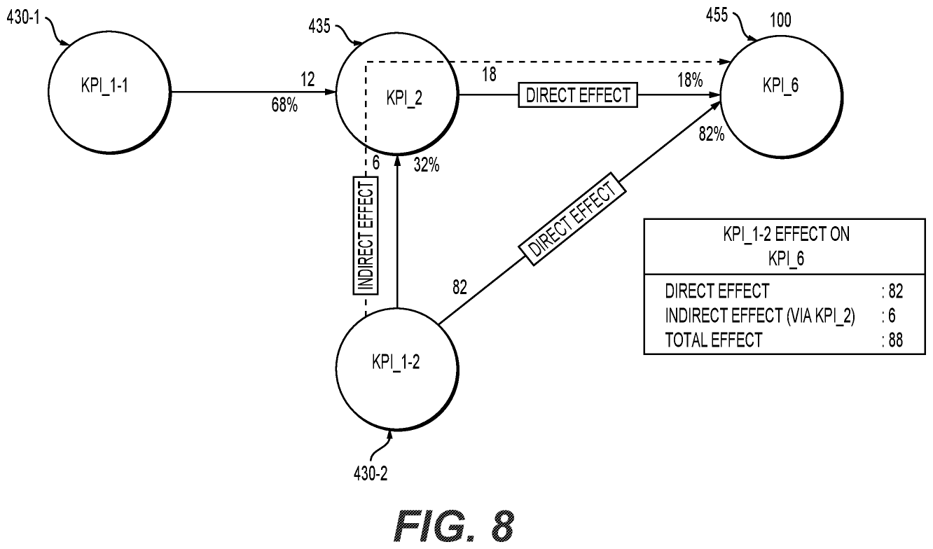

As another example, the user device 120 can display a diagram 800, as shown in FIG. 8, which is a diagram 800 of example values of directional effects between the set of KPIs. As shown in FIG. 8, the diagram 800 can display a direct directional effect, having a value of “82,” of the KPI_1-2 430-2 on the KPI_6 455. Further, the diagram 800 can display a direct directional effect, having a value of “32,” of the KPI_1-2 430-2 on the KPI_2 435, and can display a direct directional effect, having a value of “68,” of the KPI_1-1 430-1 on the KPI_2 435. Further still, the diagram 800 can display an indirect directional effect, having a value of “6,” of the KPI_1-2 430-2 on the KPI_6 455 via the KPI_2 435.

In some implementations, the platform 110 can use the values of the directional effects in association with various models to predict and forecast values of the KPIs. For example, the platform 110 can generate an AI model using the values of the directional effects of the KPIs. In this way, the platform 110 can use the AI model to predict and forecast values of the KPIs. Moreover, the platform 110 can use the predicted and forecasted values to perform various resource allocation actions, such as allocate spend, allocate resources to call centers, allocate resources of websites, allocate resources to tools, or the like.

Although FIG. 3 depicts particular blocks and a particular sequence of blocks, it should be understood that the process 300 can include different blocks, differently arranged blocks, or differently ordered blocks.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention are practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications are made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects.

Example 1. A computer-implemented method comprising: receiving, by one or more processors, information identifying a set of key performance indicators (KPIs) associated with a healthcare product; determining, by the one or more processors, a hierarchy of the set of KPIs; determining, by the one or more processors, directional effects between the set of KPIs, based on the hierarchy; determining, by the one or more processors and using structural equation modeling, values of the directional effects; providing, by the one or more processors, the values of the directional effects; and performing, by the one or more processors, a resource allocation action based on the values of the directional effects.

Example 2. The computer-implemented method of Example 1, wherein the KPIs include at least one of online spend, offline spend, visits, tool visits, started applications, completed applications, leads, switch calls, approved applications, or attributed applications.

Example 3. The computer-implemented method of any of the preceding examples, wherein the hierarchy includes a set of levels.

Example 4. The computer-implemented method of any of the preceding examples, wherein the directional effects include one or more direct effects and one or more indirect effects.

Example 5. The computer-implemented method of any of the preceding examples, wherein the values include at least one of an estimate, a standard error, a z-value, a p-value, a standardized estimate of a latent variable, or a standardized estimate.

Example 6. The computer-implemented method of any of the preceding examples, further comprising: determining, by the one or more processors, whether the values satisfy respective thresholds; and determining, by the one or more processors, an updated hierarchy based on determining that the values do not satisfy the respective thresholds.

Example 7. The computer-implemented method of any of the preceding examples, further comprising: determining, by the one or more processors, at least one of a comparative fit index or a standardized root mean square residual based on the values of the directional effects.

Example 8. A device comprising: a memory configured to store instructions; and one or more processors configured to execute the instructions to perform operations comprising: receiving information identifying a set of key performance indicators (KPIs) associated with a healthcare product; determining a hierarchy of the set of KPIs; determining directional effects between the set of KPIs, based on the hierarchy; determining, using structural equation modeling, values of the directional effects; providing the values of the directional effects; and performing a resource allocation action based on the values of the directional effects.

Example 9. The device of Example 8, wherein the KPIs include at least one of online spend, offline spend, visits, tool visits, started applications, completed applications, leads, switch calls, approved applications, or attributed applications.

Example 10. The device of any of Examples 8-9, wherein the hierarchy includes a set of levels.

Example 11. The device of any of Examples 8-10, wherein the directional effects include one or more direct effects and one or more indirect effects.

Example 12. The device of any of Examples 8-11, wherein the values include at least one of an estimate, a standard error, a z-value, a p-value, a standardized estimate of a latent variable, or a standardized estimate.

Example 13. The device of any of Examples 8-12, wherein the operations further comprise: determining whether the values satisfy respective thresholds; and determining an updated hierarchy based on determining that the values do not satisfy the respective thresholds.

Example 14. The device of any of Examples 8-13, wherein the operations further comprise: determining at least one of a comparative fit index or a standardized root mean square residual based on the values of the directional effects.

Example 15. A non-transitory computer-readable medium configured to store instructions that, when executed by one or more processors, cause the one or more processors to perform operations comprising: receiving information identifying a set of key performance indicators (KPIs) associated with a healthcare product; determining a hierarchy of the set of KPIs; determining directional effects between the set of KPIs, based on the hierarchy; determining, using structural equation modeling, values of the directional effects; providing the values of the directional effects; and performing a resource allocation action based on the values of the directional effects.

Example 16. The non-transitory computer-readable medium of Example 15, wherein the KPIs include at least one of online spend, offline spend, visits, tool visits, started applications, completed applications, leads, switch calls, approved applications, or attributed applications.

Example 17. The non-transitory computer-readable medium of any of Examples 15-16, wherein the hierarchy includes a set of levels.

Example 18. The non-transitory computer-readable medium of any of Examples 15-17, wherein the directional effects include one or more direct effects and one or more indirect effects.

Examples 19. The non-transitory computer-readable medium of any of Examples 15-18, wherein the values include at least one of an estimate, a standard error, a z-value, a p-value, a standardized estimate of a latent variable, or a standardized estimate.

Example 20. The non-transitory computer-readable medium of any of Examples 15-19, wherein the operations further comprise: determining whether the values satisfy respective thresholds; and determining an updated hierarchy based on determining that the values do not satisfy the respective thresholds.

We claim:

1. A computer-implemented method comprising:
receiving, by one or more processors, information identifying a set of key performance indicators (KPIs) associated with a healthcare product;
determining, by the one or more processors, a hierarchy of the set of KPIs;
determining, by the one or more processors, directional effects between the set of KPIs, based on the hierarchy;
determining, by the one or more processors and using structural equation modeling, values of the directional effects;
providing, by the one or more processors, the values of the directional effects; and
performing, by the one or more processors, a resource allocation action based on the values of the directional effects.

2. The computer-implemented method of claim 1, wherein the KPIs include at least one of online spend, offline spend, visits, tool visits, started applications, completed applications, leads, switch calls, approved applications, or attributed applications.

3. The computer-implemented method of claim 1, wherein the hierarchy includes a set of levels.

4. The computer-implemented method of claim 1, wherein the directional effects include one or more direct effects and one or more indirect effects.

5. The computer-implemented method of claim 1, wherein the values include at least one of an estimate, a standard error, a z-value, a p-value, a standardized estimate of a latent variable, or a standardized estimate.

6. The computer-implemented method of claim 1, further comprising:
determining, by the one or more processors, whether the values satisfy respective thresholds; and
determining, by the one or more processors, an updated hierarchy based on determining that the values do not satisfy the respective thresholds.

7. The computer-implemented method of claim 1, further comprising:
determining, by the one or more processors, at least one of a comparative fit index or a standardized root mean square residual based on the values of the directional effects.

8. A device comprising:
a memory configured to store instructions; and
one or more processors configured to execute the instructions to perform operations comprising:
receiving information identifying a set of key performance indicators (KPIs) associated with a healthcare product;
determining a hierarchy of the set of KPIs;
determining directional effects between the set of KPIs, based on the hierarchy;
determining, using structural equation modeling, values of the directional effects;
providing the values of the directional effects; and
performing a resource allocation action based on the values of the directional effects.

9. The device of claim 8, wherein the KPIs include at least one of online spend, offline spend, visits, tool visits, started applications, completed applications, leads, switch calls, approved applications, or attributed applications.

10. The device of claim 8, wherein the hierarchy includes a set of levels.

11. The device of claim 8, wherein the directional effects include one or more direct effects and one or more indirect effects.

12. The device of claim 8, wherein the values include at least one of an estimate, a standard error, a z-value, a p-value, a standardized estimate of a latent variable, or a standardized estimate.

13. The device of claim 8, wherein the operations further comprise:
determining whether the values satisfy respective thresholds; and
determining an updated hierarchy based on determining that the values do not satisfy the respective thresholds.

14. The device of claim 8, wherein the operations further comprise:
determining at least one of a comparative fit index or a standardized root mean square residual based on the values of the directional effects.

15. A non-transitory computer-readable medium configured to store instructions that, when executed by one or more processors, cause the one or more processors to perform operations comprising:
receiving information identifying a set of key performance indicators (KPIs) associated with a healthcare product;
determining a hierarchy of the set of KPIs;
determining directional effects between the set of KPIs, based on the hierarchy;
determining, using structural equation modeling, values of the directional effects;
providing the values of the directional effects; and
performing a resource allocation action based on the values of the directional effects.

16. The non-transitory computer-readable medium of claim 15, wherein the KPIs include at least one of online spend, offline spend, visits, tool visits, started applications, completed applications, leads, switch calls, approved applications, or attributed applications.

17. The non-transitory computer-readable medium of claim 15, wherein the hierarchy includes a set of levels.

18. The non-transitory computer-readable medium of claim 15, wherein the directional effects include one or more direct effects and one or more indirect effects.

19. The non-transitory computer-readable medium of claim 15, wherein the values include at least one of an estimate, a standard error, a z-value, a p-value, a standardized estimate of a latent variable, or a standardized estimate.

20. The non-transitory computer-readable medium of claim 15, wherein the operations further comprise:
determining whether the values satisfy respective thresholds; and
determining an updated hierarchy based on determining that the values do not satisfy the respective thresholds.
